(19) United States
(12) Patent Application Publication
(10) Pub. No.: US 2026/0062753 A1
(43) Pub. Date: Mar. 5, 2026

(54) CIRCULATING SERUM MICRORNA BIOMARKERS AND METHODS FOR PARKINSON'S DISEASE PROGNOSIS

(71) Applicant: ST. JOHN'S UNIVERSITY, Queens, NY (US)

(72) Inventors: Simon Geir Moller, Queens, NY (US); Indranil Basak, Queens, NY (US); Ketan Patil, Queens, NY (US); Jan Petter Larsen, Queens, NY (US); Guido Werner Alves, Queens, NY (US)

(21) Appl. No.: 19/295,990

(22) Filed: Aug. 11, 2025

Related U.S. Application Data
(62) Division of application No. 18/452,778, filed on Aug. 21, 2023, now Pat. No. 12,448,653.
Application No. 18/452,778 is a division of application No. 16/623,870, now Pat. No. 11,739,384.
(60) Provisional application No. 62/521,797, filed on Jun. 19, 2017.

(51) Int. Cl. C12Q 1/6883

(52) U.S. Cl. CPC C12Q 1/6883; C12Q 2600/112; C12Q 2600/158; C12Q 2600/178

(57) ABSTRACT

Biomarkers and methods for identifying, verifying and confirming circulating serum-based microRNAs. The microRNAs (PrognomiRs) can be used to differentiate patient's suffering from rapid progressing Parkinson's disease (PD) from slow progressing PD patients.

This application is a divisional of U.S. patent application Ser. No. 18/452,778 filed Aug. 21, 2023 which is a divisional of U.S. patent application Ser. No. 16/623,870 filed Dec. 18, 2019 issued as U.S. Pat. No. 11,739,384 on Aug. 29, 2023, which claims benefit of PCT Application No. PCT/US2018/036380 filed Jun. 7, 2018, which in turn claims benefit of U.S. Provisional Patent Application No. 62/521,797 filed Jun. 19, 2017, which are hereby incorporated by reference herein in their entirety.

### BACKGROUND OF THE INVENTION

The instant application contains a Sequence Listing which has been submitted electronically in XML file format and is hereby incorporated by reference in its entirety. Said XML copy, created on Aug. 16, 2023, is named 140415_614720_SL.xml and is 50,367 bytes in size.

### 1. FIELD OF THE INVENTION

The present invention generally relates to serum-based microRNAs and methods for differentiating patients suffering from Parkinson's disease based on the rate of disease progression, as well as assisting clinicians to determine treatment protocols for such patients.

### 2. BRIEF DESCRIPTION OF THE BACKGROUND ART

Parkinson's Disease (PD) is a highly specific degeneration of dopamine-containing cells of the substantia nigra of the midbrain, causing a dopamine deficiency in the striatum. PD currently affects about 10 million people world-wide. Effective management of a patient with PD is possible in the first 5-7 years of treatment, after which time a series of often debilitating complications, together referred to as Late Motor Fluctuations (LMF) occur. It is believed that treatment with levodopa ((−)-L-α-amino-beta-(3,4-dihydroxybenzene) propanoic acid), or L-dopa, the most effective antiparkinson drug, may facilitate or even promote the appearance of LMF. Dopamine agonists are employed as a treatment alternative, but they do not offer the same degree of symptomatic relief to patients as L-dopa does.

Symptomatic therapies improve signs and symptoms without affecting the underlying disease state. Levodopa increases dopamine concentration in the striatum, especially when its peripheral metabolism is inhibited by a peripheral decarboxylase inhibitor (PDI). Levodopa/PDI therapy is widely used for symptomatic therapy for Parkinson's disease, such as combinations with levodopa, with carbidopa ((−)-L-α-hydrazino-α-methyl-beta-(3,4-dihydroxybenzene) propanoic acid monohydrate), levodopa and controlled release carbidopa, levodopa and benserazide, levodopa plus controlled release benserazide (2-Amino-3-hydroxy-propionic acid N′-(2,3,4-trihydroxy-benzyl)-hydrazide).

Catechol-O-methyltransferase (COMT) inhibitors enhance levodopa treatment as they inhibit levodopa's metabolism, enhancing its bioavailability and thereby making more of the drug available in the synaptic cleft for a longer period of time. Examples of COMT inhibitors include tolcapone (3,4-dihydroxy-4′-methyl-5-nitrobenzophenone) and entacapone ((E)-2-cyano-3-(3,4-dihydroxy-5-nitrophenyl)-N,N-diethyl-2-propenamide).

Dopamine agonists provide symptomatic benefit by directly stimulating post-synaptic striatal dopamine receptors. Examples include bromocriptine ((5α)-2-Bromo-12′-hydroxy-2′-(1-methylethyl)-5′-(2-methylpropyl)erg-otaman-3′,6′,18-trione), pergolide (8B-[(Methylthio)methyl]-6-propylergoline), ropinirole (4-[2-(Dipropylamino)ethyl]-1,3-dihydro-2H-indol-2-one), pramipexole ((S)-4,5,6,7-Tetrahydro-N⁶-propyl-2,6-benzothiazolediamine), lisuride (N′-[(8a)-9,10-didehydro-6-methylergolin-8-yl]-N,N-diethyl-urea), cabergoline ((8β)-N-[3-(Dimethylamino) propyl]-N-[(ethylamino)carbonyl]-6-(2-propenyl)ergoline-8-carboxamide), apomorphine ((6aR)-5,6,6a,7-Tetrahydro-6-methyl-4H-dibenzo[de,g]quinoline-10,11-diol), sumanirole (5-(methylamino)-5,6-dihydro-4H-imidazo {4,5,1-ij}quinolin-2 (1H)-one), rotigotine ((−)(S)-5,6,7,8-tetrahydro-6-[propyl[2-(2-thienyl)ethyl]amino]-1-naphthol-), talipexole (5,6,7,8-Tetrahydro-6-(2-propenyl)-4H-thiazolo[4,5-d]azepin-2-amine), and dihydroergocriptine (ergotaman-3′,6′,18-trione,9,10-dihydro-12′-hydroxy-2′-methyl-5′-(phenylmethyl) (5′ cc)). Dopamine agonists are effective as monotherapy early in the course of Parkinson's disease and as an adjunct to levodopa in more advanced stages. Unlike levodopa, dopamine agonists directly stimulate post-synaptic dopamine receptors. They do not undergo oxidative metabolism and are not thought to accelerate the disease process.

Amantidine (1-Aminotricyclo (3,3,1,1^{3,7}) decane) is an antiviral agent that was discovered by chance to have anti-Parkinsonian activity. Its mechanism of action in PD has not been established, but is believed to work by increasing dopamine release. Patients who receive amantidine either as monotherapy or in combination with levodopa show improvement in akinesia, rigidity and tremor.

Other medications used in the treatment of Parkinson's disease include MAO-B inhibitors. Inhibition of L-dopa metabolism through inactivation of the monoamino oxidase type B (MAO-B) is an effective means of enhancing the efficacy of both endogenous residual dopamine and that exogenously derived from its precursor, L-dopa. Selegiline (methyl-(1-methyl-2-phenyl-ethyl)-prop-2-ynyl-amine) is a MAO-B inhibitor. There is evidence that treatment with selegiline may slow down disease progression in PD by blocking formation of free radicals derived from the oxidative metabolism of dopamine. Other examples of MAO B inhibitors include lazabemide (N-(2-Aminoethyl)-5-chloro-2-pyridinecarboxamide), rasagiline (N-propargyl-1-(R)aminoindan and caroxazone (2-oxo-2H-1,3-benzoxazine-3 (4H)-acetamide).

It is imperative to diagnose individuals with PD at an early stage and it is also important to determine the prognosis of the disease to increase the efficacy of therapeutic agents. However, there are neither any objective tests nor established biomarkers for diagnosing PD. Moreover, the heterogeneity, subtypes and progression of the disease make it difficult to develop specific therapeutic candidates.

MicroRNAs (“miRNAs) are a class of non-coding RNAs that play key roles in the regulation of gene expression. miRNAs act at the post-transcriptional level and fine-tune the expression of as much as 30% of all mammalian protein-encoding genes. Mature miRNAs are short, single-stranded RNA molecules approximately 22 nucleotides in length. miRNAs may be encoded by multiple loci, and may be organized in tandemly co-transcribed clusters. miRNA genes are transcribed by RNA polymerase II as large primary transcripts (pri-microRNA) that are processed by a protein complex containing the RNase III enzyme Drosha, DGCR8 and other cofactors, to form an approximately 70 nucleotide precursor microRNA (pre-miRNA). (Cathew R W, Cell, 2009; Kim V N, Nat Rev Mol Cel Biol, 2009; Siomi H, Mol Cel, 2010; Bartel D P, Cell, 2004; Lee Y, Nature 2003; Han J, Genes Dev, 2004.) Pre-miRNA is transported to the cytoplasm by Exportin-5 where it is processed by DICER, a second RNase III enzyme, together with TRBP, PACT and Ago2 in the RNA Induced Silencing Complex resulting in miRNA duplexes (Kim V N, Nat Rev Mol Cel Biol, 2009: Gregory R I, Nature 2004: MAcRae I J, PNAS, 2008). The guide strands of miRNA duplexes separate and associate with Ago 2 for incorporation into a ribonuclear particle to form the RNA-induced silencing complex RISC that mediates gene silencing. The mechanisms of miRNA range from direct degradation or silencing of mRNA and repression of translation to post-transcriptional upregulations. (MacRae I J, PNAS, 2008.)

The presence of miRNAs has been reported in body fluids including blood, cerebrospinal fluid (CSF), plasma, serum and saliva at detectable levels. The tissue-specificity of miRNAs suggests their vital and integral role in various physiological processes. The tissue-enrichment promises a new but less explored role as diagnostic biomarker and potential therapeutic target. Circulating miRNAs are understood to originate from passive leakage from damaged tissue as a result of cell lysis or apoptosis, active transport from cells via microvesicles, such as exosomes, or bound within RISC protein complexes (Etheridge et al, 2011). Exosome and osmotic pump-mediated delivery of small RNA molecules to the brain and CNS, respectively, provides a solution to overcoming the limitations of miRNA-based therapies (Alvarez-Erviti et al., 2011; Koval et al, 2013, Hum. Mol. Gen). miRNA has been demonstrated to be exceptionally stable and thus present as powerful candidates to be potential biomarkers (Chen et al, 2008; Grasso, 2014).

### SUMMARY OF THE INVENTION

It is an object of the present invention to identify miRNAs relevant to patients suffering from Parkinson's disease.

It is another object of the present invention to provide methods for determining the rate of disease progression for patients suffering from Parkinson's disease.

It is another object of the present invention to determine the methods for determining treatment option based on the rate of progression of the disease for patients suffering from Parkinson's disease.

These objects and others are achieved by the present invention, which provides miRNA biomarkers that may be used singly, in pairs or in combination to determine patients suffering from Parkinson's disease.

### BRIEF DESCRIPTION OF THE DRAWINGS

FIG. 1 shows the mean fold change of five PrognomiRNAs between rapid and slow progressing PD patients;

### DETAILED DESCRIPTION OF THE INVENTION

### Methods

### Serum Samples Handling and Classification

All patients and controls participated in the Norwegian ParkWest project, which is an ongoing prospective population-based longitudinal cohort study investigating the incidence, neurobiology and prognosis of PD. The Norwegian ParkWest study is a prospective longitudinal multicenter cohort study of patients with incident Parkinson's disease (PD) from Western and Southern Norway. Between November 1st 2004 and 31st of August 2006 it was endeavored to recruit all new cases of Parkinson Disease within the study area. Since the start of the study 212 of 265 (80%) of these patients and their age-/sex-matched control group have been followed.

All possible efforts were undertaken to establish an unselected and population-representative cohort of patients with PD. Patients were included if they had provided serum at study entry and fulfilled diagnostic criteria for PD of the National Institute of Neurological Disorders and Stroke and UK Brain Bank at latest follow-up. Patients with secondary parkinsonism at study entry were excluded from this study. Control subjects were recruited from multiple sources, including friends, spouses, and public organizations for elderly and were included in this study if they had provided serum. All patients and controls were Caucasian. The participants of the study have been followed for over eight years to establish the rate of disease progression.

In this study of possible biomarkers for PD we applied a two-stage procedure. For the first discovery phase serum from 8 patients with rapid and 8 patients with slow progression of PD were selected at random. The remaining 164 patients with PD that were eligible for this study were selected for verification purposes.

Serum samples were collected at the same day as the clinical examinations and then stored frozen at −70 degrees Celsius until transported to the facilities in New York on dry ice.

### Example 1: Analyses of Differentially Expressed Human miRNA By qPCR

RNA Isolation from Serum Samples and QC

After thawing on ice, twenty-four (sixteen PD samples) serum samples were spun down for 5 mins at 3000×g to remove debris. The supernatant was used to perform small RNA isolation using miRCURY RNA Isolation Kit-Biofluids (Exiqon, MA). Before RNA Isolation, the lysis buffer was spiked with 0.267 fmol/ul of spike-in control cel-miR-39-3p (Qiagen, CA). The remaining part of the RNA isolation was performed following manufacturer's protocol and the isolated RNA was quantified on a Nanodrop 2000 (Thermo Scientific, MA). The RNA was used for running Affymetrix v4 microRNA microarray chips and for subsequent cDNA synthesis and qPCR. RNA from 180 serum samples (from PD patients from ParkWest project) was isolated as described above, they were not quantified by Nanodrop, but the qPCR data resulting from these samples were normalized by a reference small RNA scaRNA17.

miRNA Microarray and Data Analysis

The isolated RNA from twenty-four patient serum samples were quantified and subjected to Affymetrix GeneChip® miRNA 4.0 Array by the Yale Center for Genome Analysis. The normalized .CEL files obtained from Affymetrix Expression Console software were imported into Partek Genomics Suite version 6.6 Copyright © 2012 (Partek, MO) for analysis. The ‘microRNA Expression Workflow’ was employed to detect differentially expressed miRNAs employing ANOVA resulting in lists of miRNAs significantly (p<0.05) expressed between rapid and slow PD cohorts. The miRNAs detected were used for further qPCR verification.

### Quantitative Polymerase Chain Reaction

cDNA for miRNA specific qPCR was synthesized using qScript™ microRNA cDNA Synthesis kit (Quanta Biosciences, MD) following manufacturer's protocol and subsequent qPCRs were performed using miRNA specific forward primers (Table #) and PerfeCTa®Universal PCR primer (Quanta Biosciences, MD). scaRNA17 and U6 were used reference small RNAs for normalizing qPCR Cq values whereas cel-miR-39-3p was used as spike-in control. PerfeCTa® SYBR& GREEN SuperMix for IQ™ (Quanta Biosciences, MD) was used for all qPCRs in a MyiQ™ Single color Real-Time PCR Detection System (Bio-Rad, CA). Standard curve for cel-miR-39-3p was analyzed in MS Excel with R²=0.97882 and PCR efficiency 92.96%. No Template Control (NTC) was implied wherever needed.

Differentially expressed human miRNAs in Parkinson's disease patients' serum samples from The Norwegian Park West study were determined employing miRNA microarray. Provided below are the miRNAs with >1.2 fold differential expression.

52 Differentially Expressed Human Pre- and Mature miRNAs With >1.2 Fold Change

hsa-miR-6865-3p, hsa-miR-663a, hsa-miR-92b-3p, hsa-miR-455-3p, hsa-miR-937-5p, hsa-let-7b-3p, hsa-miR-6730-3p, hsa-miR-5010-3p, hsa-miR-1825, hsa-mir-4487, hsa-miR-4783-5p, hsa-miR-2117, hsa-mir-5090, hsa-mir-4484, hsa-miR-5094, hsa-mir-611, hsa-miR-4738-3p, hsa-miR-6894-5p, hsa-mir-8072, hsa-mir-762, hsa-let-7e, hsa-miR-6768-5p, hsa-mir-3917, hsa-mir-3673, hsa-mir-4431, hsa-miR-216a-3p, hsa-miR-635, hsa-miR-490-3p, hsa-mir-601, hsa-miR-636, hsa-miR-466, hsa-miR-1271-5p, hsa-miR-548u, hsa-miR-3606-5p, hsa-miR-510-5p, hsa-miR-4306, hsa-mir-4753, hsa-mir-6128, hsa-mir-4251, hsa-miR-1306-5p, hsa-miR-8052, hsa-mir-4310, hsa-mir-3128, hsa-miR-628-5p, hsa-miR-3660, hsa-miR-3156-3p, hsa-miR-548aj-3p, hsa-miR-4791, hsa-mir-532, hsa-miR-202-5p, hsa-miR-3613-3p, hsa-miR-8075

36 differentially expressed mature miRNAs with >1.2 fold change hsa-miR-6865-3p, hsa-miR-663a, hsa-miR-92b-3p, hsa-miR-455-3p, hsa-miR-937-5p, hsa-let-7b-3p, hsa-miR-6730-3p, hsa-miR-5010-3p, hsa-miR-1825, hsa-miR-4783-5p, hsa-miR-2117, hsa-miR-5094, hsa-miR-4738-3p, hsa-miR-6894-5p, hsa-let-7e, hsa-miR-6768-5p, hsa-miR-216a-3p, hsa-miR-635, hsa-miR-490-3p, hsa-miR-636, hsa-miR-466, hsa-miR-1271-5p, hsa-miR-548u, hsa-miR-3606-5p, hsa-miR-510-5p, hsa-miR-4306, hsa-miR-1306-5p, hsa-miR-8052, hsa-miR-628-5p, hsa-miR-3660, hsa-miR-3156-3p, hsa-miR-548aj-3p, hsa-miR-4791, hsa-miR-202-5p, hsa-miR-3613-3p, hsa-miR-8075

16 differentially expressed premature miRNAs with >1.2 fold change hsa-mir-4487, hsa-mir-5090, hsa-mir-4484, hsa-mir-611, hsa-mir-8072, hsa-mir-762, hsa-mir-3917, hsa-mir-3673, hsa-mir-4431, hsa-mir-601, hsa-mir-4753, hsa-mir-6128, hsa-mir-4251, hsa-mir-4310, hsa-mir-3128, hsa-mir-532, These differentially expressed miRNA sequences are illustrated below in Table 1, along with the reference/house-keeping small RNAs cel-miR-39-3p, U6 and ScaRNA17 used as controls. Cel-miR-39-3p is a spike-in control that demonstrates the stability of the RNA samples. U6 and ScaRNA17 are used as internal controls to normalize the readings of the rest of the miRNAs or candidate miRNAs.

### Example 1

TABLE 1

| microRNA/small RNA name | microRNA Sequence |
|---|---|
| hsa-let-7b-3p | CUAUACAACCUACUGCCUUCCC (SEQ ID NO: 1) |
| hsa-let-7e | CCCGGGCUGAGGUAGGAGGUUGUAUAGUUGAGGAGGACACCC |
| | AAGGAGAUCACUAUACGGCCUCCUAGCUUUCCCCAGG |
| | (SEQ ID NO: 2) |
| hsa-miR-1271-5p | CUUGGCACCUAGCAAGCACUCA (SEQ ID NO: 3) |
| hsa-miR-1306-5p | CCACCUCCCCUGCAAACGUCCA (SEQ ID NO: 4) |
| hsa-miR-1825 | UCCAGUGCCCUCCUCUCC (SEQ ID NO: 5) |
| hsa-miR-202-5p | UUCCUAUGCAUAUACUUCUUUG (SEQ ID NO: 6) |
| hsa-miR-2117 | UGUUCUCUUUGCCAAGGACAG (SEQ ID NO: 7) |
| hsa-miR-216a-3p | UCACAGUGGUCUCUGGGAUUAU (SEQ ID NO: 8) |
| hsa-mir-3128 | UUCCUCUGGCAAGUAAAAAACUCUCAUUUUCCUUAAAAAAUG |
| | AGAGUUUUUUACUUGCAAUAGGAA (SEQ ID NO: 9) |
| hsa-miR-3156-3p | CUCCCACUUCCAGAUCUUUCU (SEQ ID NO: 10) |
| hsa-miR-3606-5p | UUAGUGAAGGCUAUUUUAAUU (SEQ ID NO: 11) |
| hsa-miR-3613-3p | ACAAAAAAAAAAGCCCAACCCUUC (SEQ ID NO: 12) |
| hsa-miR-3660 | ACUGACAGGAGAGCAUUUUGA (SEQ ID NO: 13) |
| hsa-mir-3673 | AUAUAUAUAUAUGGAAUGUAUAUACGGAAUAUAUAUAUAUA |
| | UGGAAUGUAUAUACGGAAUAUAUAUAUAUAUGGAAUGUAUU |
| | ACGGAAUAUAUAUAUAU |
| | AU (SEQ ID NO: 14) |
| hsa-mir-3917 | GGCGCUUUUGUGCGCGCCCGGGUCUGUUGGUGCUCAGAGUGU |
| | GGUCAGGCGGCUCGGACUGAGCAGGUGGGUGCGGGGCUCGGA |
| | GGAGGCGGC (SEQ ID NO: 15) |
| hsa-mir-4251 | CACGUCCUCCAGCUUUUUUCCUUAGUGGCCAAUUCCUGAGAA |
| | AAGGGCCAACGUGCUUCCA (SEQ ID NO: 16) |
| hsa-miR-4306 | UGGAGAGAAAGGCAGUA (SEQ ID NO: 17) |
| hsa-mir-4310 | UGGCGUCUGGGGCCUGAGGCUGCAGAACAUUGCAGCAUUCAU |
| | GUCCCACCCCCACCA (SEQ ID NO: 18) |
| hsa-mir-4431 | UGGUUUGCGACUCUGAAAACUAGAAGGUUUAUGACUGGGCA |
| | UUUCUCACCCAAUGCCCAAUAUUGAACUUUCUAGUUGUCAGA |
| | GUCAUUAACCC (SEQ ID NO: 19) |
| hsa-mir-4484 | GGGUUUCCUCUGCCUUUUUUUCCAAUGAAAAUAACGAAACCU |
| | GUUAUUUCCCAUUGAGGGGGAAAAAGGCGGGAGAAGCCCCA |
| | (SEQ ID NO: 20) |
| hsa-mir-4487 | ACUGUCCUUCAGCCAGAGCUGGCUGAAGGGCAGAAGGGAACU |
| | GUCCUUCAGCCAGAGCUGGCUGAAGGGCAGA (SEQ ID NO: 21) |
| hsa-miR-455-3p | GCAGUCCAUGGGCAUAUACAC (SEQ ID NO: 22) |
| hsa-miR-466 | AUACACAUACACGCAACACACAU (SEQ ID NO: 23) |
| hsa-miR-4738-3p | UGAAACUGGAGCGCCUGGAGGA (SEQ ID NO: 24) |
| hsa-mir-4753 | AUAUCUACACAAGGCCAAAGGAAGAGAACAGAUAUAUCCACA |
| | GUACACUUGGCUGUUCUCUUUCUUUAGCCUUGUGUAGAUAU |
| | (SEQ ID NO: 25) |
| hsa-miR-4783-5p | GGCGCGCCCAGCUCCCGGGCU (SEQ ID NO: 26) |
| hsa-miR-4791 | UGGAUAUGAUGACUGAAA (SEQ ID NO: 27) |
| hsa-miR-490-3p | CAACCUGGAGGACUCCAUGCUG (SEQ ID NO: 28) |
| hsa-miR-5010-3p | UUUUGUGUCUCCCAUUCCCCAG (SEQ ID NO: 29) |
| hsa-mir-5090 | UCUGAGGUACCCGGGGCAGAUUGGUGUAGGGUGCAAAGCCUG |
| | CCCGCCCCCUAAGCCUUCUGCCCCCAACUCCAGCCUGUCAGGA |
| | (SEQ ID NO: 30) |
| hsa-miR-5094 | AAUCAGUGAAUGCCUUGAACCU (SEQ ID NO: 31) |
| hsa-miR-510-5p | UACUCAGGAGAGUGGCAAUCAC (SEQ ID NO: 32) |
| hsa-mir-532 | CGACUUGCUUUCUCUCCUCCAUGCCUUGAGUGUAGGACCGUU |
| | GGCAUCUUAAUUACCCUCCCACACCCAAGGCUUGCAGAAGAG |
| | CGAGCCU (SEQ ID NO: 33) |
| hsa-miR-548aj-3p | UAAAAACUGCAAUUACUUUUA (SEQ ID NO: 34) |
| hsa-miR-548u | CAAAGACUGCAAUUACUUUUGCG (SEQ ID NO: 35) |
| hsa-mir-601 | UGCAUGAGUUCGUCUUGGUCUAGGAUUGUUGGAGGAGUCAG |
| | AAAAACUACCCCAGGGAUCCUGAAGUCCUUUGGGUGGA |
| | (SEQ ID NO: 36) |
| hsa-mir-611 | AAAAUGGUGAGAGCGUUGAGGGGAGUUCCAGACGGAGAUGC |
| | GAGGACCCCUCGGGGUCUGACCCACA (SEQ ID NO: 37) |
| hsa-mir-6128 | AAGAAGCUUGUAGAUUUUUCUCCCUUACUAUCUAGAAUUAUA |
| | GGACUUCAGUCCAUGAUUUGGAAAAAUUACUGGAAUUGGAG |
| | UCAAAAAUAAUUUGAAAAUUAGGAAU (SEQ ID NO: 38) |
| hsa-miR-628-5p | AUGCUGACAUAUUUACUAGAGG (SEQ ID NO: 39) |
| hsa-miR-635 | ACUUGGGCACUGAAACAAUGUCC (SEQ ID NO: 40) |
| hsa-miR-636 | UGUGCUUGCUCGUCCCGCCCGCA (SEQ ID NO: 41) |
| hsa-miR-663a | AGGCGGGGCGCCGCGGGACCGC (SEQ ID NO: 42) |
| hsa-miR-6730-3p | CCUGACACCCCAUCUGCCCUCA (SEQ ID NO: 43) |
| hsa-miR-6768-5p | CACACAGGAAAAGCGGGGCCCUG (SEQ ID NO: 44) |
| hsa-miR-6865-3p | ACACCCUCUUUCCCUACCGCC (SEQ ID NO: 45) |
| hsa-miR-6894-5p | AGGAGGAUGGAGAGCUGGGCCAGA (SEQ ID NO: 46) |
| hsa-mir-762 | GGCCCGGCUCCGGGUCUCGGCCCGUACAGUCCGGCCGGCCAU |
| | GCUGGCGGGGCUGGGGCCGGGGCCGAGCCCGCGGCGGGGCC |
| | (SEQ ID NO: 47) |
| hsa-miR-8052 | CGGGACUGUAGAGGGCAUGAGC (SEQ ID NO: 48) |
| hsa-mir-8072 | GCGUCAAGAUGGCGGCGGGGAGGUAGGCAGAGCAGGACGCCG |
| | CUGCUGCCGCCGCCACCGCCGCCUCCGCUCCAGUCGCC |
| | (SEQ ID NO: 49) |
| hsa-miR-8075 | UGCUGAUGGCAGAUGUCGGGUCUG |
| | (SEQ ID NO: 50) |
| hsa-miR-92b-3p | UAUUGCACUCGUCCCGGCCUCC (SEQ ID NO: 51) |
| hsa-miR-937-5p | GUGAGUCAGGGUGGGGCUGG (SEQ ID NO: 52) |
| cel-miR-39-3p | UCACCGGGUGUAAAUCAGCUUG (SEQ ID NO: 53) |
| scaRNA17 | AGAGGCUUGGGCCGCCGAGCUGGACCCGGACCGGUUUUGGGU |
| | ACUGUACUGGGGGCAGGGCAGAGAGGG (SEQ ID NO: 54) |
| U6 | GUGCUCGCUUCGGCAGCACAUAUACUAAAAUUGGAACGAUAC |
| | AGAGAAGAUUAGCAUGGCCCCUGCGCAAGGAUGACACGCAAA |
| | UUCGUGAAGCGUUCCAUAUUUU (SEQ ID NO: 55) |

### Example 2: Verification of Human Mature miRNAs by qPCR in Sample Cohort of 8 Rapid and 8 Slow Progressing Patients

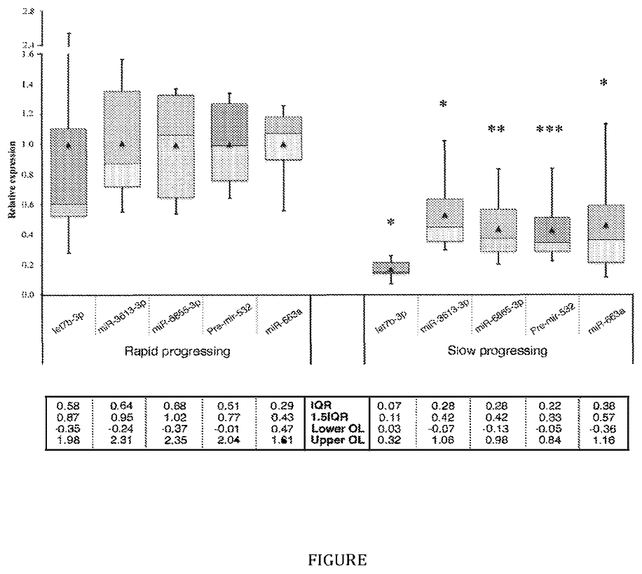

The mean fold change for hsa-let7b-3p, hsa-miR-3613-3p and hsa-miR-6865-3p, hsa-pre-miR-532, and hsa-miR-663a, PrognomiRs between rapid and slow progressing PD patients are shown below in Table 2 and illustrated in FIG. 1.

TABLE 2

| PrognomiR | Fold change | Significance |
|---|---|---|
| hsa-let7b-3p | 0.17 | 0.02 |
| hsa-miR-3613-3p | 0.53 | 0.01 |
| hsa-miR-6865-3p | 0.44 | 0.002 |
| hsa-pre-miR-532 | 0.43 | 0.0005 |
| hsa-miR-663a | 0.46 | 0.01 |

### Example 3

Measurement of levels of a combination of two or more miRNAs in serum from patients can assist in distinctly differentiating between a potential rapid as opposed to slow progressing PD patient. A serum sample is obtained from blood withdrawn from patients suspected of PD. The serum is used for total microRNA isolation and enrichment. This RNA would then be tested using qPCR to measure the levels of any two or more of the 52 miRNAs mentioned in Example 1, or any one of five miRNAs mentioned in Example 2. Detectable levels of one or more of the aforementioned 52 miRNAs with >1.2 fold change, or any one or more of these five miRNAs confirms the rate of progression for patients with PD. If desired, other sample fluids may be utilized, including plasma, venous or arterial blood, or CSF samples withdrawn by lumbar puncture. Such plasma, blood or CSF samples are processed as discussed above regarding serum, e.g., so as to provide a sample for processing and evaluation outside the human or animal body. It will be understood that measurement of more than two miRNAs in combination or a set of combinations used in a test matrix may desirably increase the accuracy of predicting PD progression. Following diagnosis, the result is then communicated to the patient.

As discussed and illustrated above, detecting the aforementioned miRNAs with >1.2 fold change may be used to differentiate potential rapid progressing PD from potential slow progressing PD in afflicted patients. However, as readily understood by those of ordinary skill herein, other thresholds may be suitably employed as desired with possible concomitant variation in accuracy, including >1.0, 1.1, 1.3, 1.4, 1.5, 1.6, 1.7, 1.8, 1.9, and 2.0 fold changes. Moreover, it will be similarly understood that these varying fold change thresholds may be more suitably used with particular miRNAs without affecting accuracy.

### Example 4

Since a combination of miRNA can be used for predicting prognosis it may be advisable to test all the candidates to eliminate any cohort-based variation. It is understood that any detectable amounts of relevant miRNA will indicate PD pathology. However, those of ordinary skill in the art recognize it may be clinically helpful to use values of slow (8) v rapid (8) samples to set an artificial threshold for determination of rate of disease progression. Differential miRNA levels can be used to develop prognostic biomarker kits that can be used by clinicians for estimating prognosis of the disease as well as enriching patient cohorts in clinical trials. In this study the presence and quantification of miRNA from serum was determined by qRT-PCR which amplifies and quantifies the RNA is question. Other suitable techniques known to those of ordinary skill herein may be alternatively utilized, including use of labeled antisense sequences and labeled antibodies. Suitable antibodies are preferentially selective, referring to a binding reaction between two molecules that is typically more than 10 to 100 times background molecular associations under measurement conditions. Thus, under designated immunoassay conditions, the specified antibodies bind to a particular miRNA sequence, thereby identifying its presence. Specific binding to an antibody under such conditions requires an antibody that is selected for its specificity for a particular miRNA. For example, antibodies raised against a particular miRNA can be selected by subtracting out antibodies that cross-react with other molecules. A variety of immunoassay formats may be used to select antibodies specifically immunoreactive with a particular miRNA including solid-phase ELISA immunoassay's (see, e.g., Harlow & Lane, Antibodies, A Laboratory Manual (1988) for a description of immunoassay formats and conditions that can be used to determine specific immunoreactivity). Methods for determining whether two molecules specifically interact are disclosed therein, and methods of determining binding affinity and specificity are well known in the art (see, for example, Harlow and Lane, Antibodies: A laboratory manual (Cold Spring Harbor Laboratory Press, 1988): Friefelder, “Physical Biochemistry: Applications to biochemistry and molecular biology” (W. H. Freeman and Co. 1976)). The term “antibody” as used herein encompasses naturally occurring antibodies as well as non-naturally occurring antibodies, including, for example, single chain antibodies, chimeric, bifunctional and humanized antibodies, as well as antigen-binding fragments thereof, (e.g., Fab′, F(ab′)2, Fab, Fv and rIgG). See also, Pierce Catalog and Handbook, 1994-1995 (Pierce Chemical Co., Rockford, IL). See also, e.g., Kuby, J., Immunology, 3rd Ed., W. H. Freeman & Co., New York (1998). Such non-naturally occurring antibodies can be constructed using solid phase peptide synthesis, can be produced recombinantly or can be obtained, for example, by screening combinatorial libraries consisting of variable heavy chains and variable light chains as described by Huse et al., Science, Vol. 246 (1989) 1275-81. These and other methods of making, for example, chimeric, humanized, CDR-grafted, single chain, and bifunctional antibodies are well known to those skilled in the art (Winter and Harris, Immunol. Today, Vol. 14 (1993) 243-46; Ward et al., Nature, Vol. 341 (1989) 544-46; Harlow and Lane, supra, 1988; Hilyard et al., Protein Engineering: A practical approach (IRL Press 1992); Borrabeck, Antibody Engineering, 2d ed. (Oxford University Press 1995). Methods for producing both monoclonal and polyclonal antibodies from identified RNA sequences are well known in the art.

### Example 5

Many neurodegenerative diseases are closely related to each other when it comes to symptoms as well as pathological markers. The circulating prognostic markers for one neurodegenerative disease can be useful for diagnosis/prognosis of other disease. A method to diagnose/prognoses other neurodegenerative diseases like Dementia with Lewy body (DLB), Amyotrophic lateral sclerosis (ALS), Alzheimer's disease (AD), Multiple system atrophy (MSA), CorticoBasal Degeneration (CBD), Progressive Supranuclear Palsy (PSP) can also be developed using similar miRNA measurements of candidates mentioned above. Disease specific kits can be developed similar to one mentioned with various combinations of miRNAs listed.

### Example 6

The miRNAs detected in one or more combinations can regulate several proteins in the cells. Novel protein targets for PD can be discovered using these microRNAs and their combinations. The involvement of these proteins in PD etiology can be further established to target them for therapy.

### Example 7

Small nucleic acid molecules derived from miRNAs mentioned will be designed to therapeutically intervene by specifically targeting genes in PD brains to achieve complete or partial remedy.

1-40. (canceled)

41. A method for treating a human patient with rapid progressing Parkinson's disease, comprising the steps of, in this order:
obtaining a first sample comprising saliva from said patient;
determining a level of expression of at least one miRNA selected from the group consisting of SEQ ID NOS: 33, 42 and 45, and optionally further determining a level of expression of at least one miRNA selected from the group consisting of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, 41, 43, 44, 46, 47, 48, 49, 50, 51 and 52 within said first sample;
comparing the level of expression of the determined SEQ ID NOS to a level of expression of the determined SEQ ID NOS in a second sample from a cohort with slow progressing Parkinson's disease and confirming the expression level of the determined SEQ ID NOS in said first sample is greater than 1.2-fold over that of said second sample, thereby indicating the patient has rapid progressing Parkinson's disease; and
treating said rapid progressing Parkinson's disease with at least one of L-dopa, a dopamine agonist, a catechol-O-methyltransferase (COMT) inhibitor, amantidine (1-Aminotricyclo (3,3,1,1^{3,7}) decane or a monoamino oxidase type B inhibitor.

42. The method according to claim 41, wherein expression of miRNAs according to at least SEQ ID NO: 33 is compared relative to the cohort.

43. The method according to claim 41, wherein expression of miRNAs according to at least SEQ ID NO: 42 is compared relative to the cohort.

44. The method according to claim 41, wherein expression of miRNAs according to at least SEQ ID NO: 45 is compared relative to the cohort.

45. The method according to claim 41, wherein expression of miRNAs according to at least two of said SEQ ID NOS: 33, 42 and 45 is compared relative to the cohort.

46. The method according to claim 45, wherein expression of miRNAs according to at least SEQ ID NOS: 33 and 42 is compared relative to the cohort.

47. The method according to claim 45, wherein expression of miRNAs according to at least SEQ ID NOS: 33 and 45 is compared relative to the cohort.

48. The method according to claim 45, wherein expression of miRNAs according to at least SEQ ID NOS: 42 and 45 is compared relative to the cohort.

49. The method according to claim 41, wherein expression of miRNAs according to each of said SEQ ID NOS: 33, 42 and 45 is compared relative to the cohort.


SEQUENCE LISTING (55 sequences)

SEQ ID NO: 1 - RNA, 22 nt, Homo sapiens
  source 1..22: mol_type = unassigned RNA
ctatacaacctactgccttccc

SEQ ID NO: 2 - RNA, 79 nt, Homo sapiens
  source 1..79: mol_type = unassigned RNA
cccgggctgaggtaggaggttgtatagttgaggaggacacccaaggagatcactatacggcctcctagctttccccagg

SEQ ID NO: 3 - RNA, 22 nt, Homo sapiens
  source 1..22: mol_type = unassigned RNA
cttggcacctagcaagcactca

SEQ ID NO: 4 - RNA, 22 nt, Homo sapiens
  source 1..22: mol_type = unassigned RNA
ccacctcccctgcaaacgtcca

SEQ ID NO: 5 - RNA, 18 nt, Homo sapiens
  source 1..18: mol_type = unassigned RNA
tccagtgccctcctctcc

SEQ ID NO: 6 - RNA, 22 nt, Homo sapiens
  source 1..22: mol_type = unassigned RNA
ttcctatgcatatacttctttg

SEQ ID NO: 7 - RNA, 21 nt, Homo sapiens
  source 1..21: mol_type = unassigned RNA
tgttctctttgccaaggacag

SEQ ID NO: 8 - RNA, 22 nt, Homo sapiens
  source 1..22: mol_type = unassigned RNA
tcacagtggtctctgggattat

SEQ ID NO: 9 - RNA, 66 nt, Homo sapiens
  source 1..66: mol_type = unassigned RNA
ttcctctggcaagtaaaaaactctcattttccttaaaaaatgagagttttttacttgcaataggaa

SEQ ID NO: 10 - RNA, 21 nt, Homo sapiens
  source 1..21: mol_type = unassigned RNA
ctcccacttccagatctttct

SEQ ID NO: 11 - RNA, 21 nt, Homo sapiens
  source 1..21: mol_type = unassigned RNA
ttagtgaaggctattttaatt

SEQ ID NO: 12 - RNA, 24 nt, Homo sapiens
  source 1..24: mol_type = unassigned RNA
acaaaaaaaaaagcccaacccttc

SEQ ID NO: 13 - RNA, 21 nt, Homo sapiens
  source 1..21: mol_type = unassigned RNA
actgacaggagagcattttga

SEQ ID NO: 14 - RNA, 101 nt, Homo sapiens
  source 1..101: mol_type = unassigned RNA
atatatatatatggaatgtatatacggaatatatatatatatggaatgtatatacggaatatatatatatatggaatgtattacggaatatatatatatat

SEQ ID NO: 15 - RNA, 93 nt, Homo sapiens
  source 1..93: mol_type = unassigned RNA
ggcgcttttgtgcgcgcccgggtctgttggtgctcagagtgtggtcaggcggctcggactgagcaggtgggtgcggggctcggaggaggcggc

SEQ ID NO: 16 - RNA, 61 nt, Homo sapiens
  source 1..61: mol_type = unassigned RNA
cacgtcctccagcttttttccttagtggccaattcctgagaaaagggccaacgtgcttcca

SEQ ID NO: 17 - RNA, 17 nt, Homo sapiens
  source 1..17: mol_type = unassigned RNA
tggagagaaaggcagta

SEQ ID NO: 18 - RNA, 57 nt, Homo sapiens
  source 1..57: mol_type = unassigned RNA
tggcgtctggggcctgaggctgcagaacattgcagcattcatgtcccacccccacca

SEQ ID NO: 19 - RNA, 94 nt, Homo sapiens
  source 1..94: mol_type = unassigned RNA
tggtttgcgactctgaaaactagaaggtttatgactgggcatttctcacccaatgcccaatattgaactttctagttgtcagagtcattaaccc

SEQ ID NO: 20 - RNA, 83 nt, Homo sapiens
  source 1..83: mol_type = unassigned RNA
gggtttcctctgcctttttttccaatgaaaataacgaaacctgttatttcccattgagggggaaaaaggcgggagaagcccca

SEQ ID NO: 21 - RNA, 73 nt, Homo sapiens
  source 1..73: mol_type = unassigned RNA
actgtccttcagccagagctggctgaagggcagaagggaactgtccttcagccagagctggctgaagggcaga

SEQ ID NO: 22 - RNA, 21 nt, Homo sapiens
  source 1..21: mol_type = unassigned RNA
gcagtccatgggcatatacac

SEQ ID NO: 23 - RNA, 23 nt, Homo sapiens
  source 1..23: mol_type = unassigned RNA
atacacatacacgcaacacacat

SEQ ID NO: 24 - RNA, 22 nt, Homo sapiens
  source 1..22: mol_type = unassigned RNA
tgaaactggagcgcctggagga

SEQ ID NO: 25 - RNA, 83 nt, Homo sapiens
  source 1..83: mol_type = unassigned RNA
atatctacacaaggccaaaggaagagaacagatatatccacagtacacttggctgttctctttctttagccttgtgtagatat

SEQ ID NO: 26 - RNA, 21 nt, Homo sapiens
  source 1..21: mol_type = unassigned RNA
ggcgcgcccagctcccgggct

SEQ ID NO: 27 - RNA, 18 nt, Homo sapiens
  source 1..18: mol_type = unassigned RNA
tggatatgatgactgaaa

SEQ ID NO: 28 - RNA, 22 nt, Homo sapiens
  source 1..22: mol_type = unassigned RNA
caacctggaggactccatgctg

SEQ ID NO: 29 - RNA, 22 nt, Homo sapiens
  source 1..22: mol_type = unassigned RNA
ttttgtgtctcccattccccag

SEQ ID NO: 30 - RNA, 85 nt, Homo sapiens
  source 1..85: mol_type = unassigned RNA
tctgaggtacccggggcagattggtgtagggtgcaaagcctgcccgccccctaagccttctgcccccaactccagcctgtcagga

SEQ ID NO: 31 - RNA, 22 nt, Homo sapiens
  source 1..22: mol_type = unassigned RNA
aatcagtgaatgccttgaacct

SEQ ID NO: 32 - RNA, 22 nt, Homo sapiens
  source 1..22: mol_type = unassigned RNA
tactcaggagagtggcaatcac

SEQ ID NO: 33 - RNA, 91 nt, Homo sapiens
  source 1..91: mol_type = unassigned RNA
cgacttgctttctctcctccatgccttgagtgtaggaccgttggcatcttaattaccctcccacacccaaggcttgcagaagagcgagcct

SEQ ID NO: 34 - RNA, 21 nt, Homo sapiens
  source 1..21: mol_type = unassigned RNA
taaaaactgcaattactttta

SEQ ID NO: 35 - RNA, 23 nt, Homo sapiens
  source 1..23: mol_type = unassigned RNA
caaagactgcaattacttttgcg

SEQ ID NO: 36 - RNA, 79 nt, Homo sapiens
  source 1..79: mol_type = unassigned RNA
tgcatgagttcgtcttggtctaggattgttggaggagtcagaaaaactaccccagggatcctgaagtcctttgggtgga

SEQ ID NO: 37 - RNA, 67 nt, Homo sapiens
  source 1..67: mol_type = unassigned RNA
aaaatggtgagagcgttgaggggagttccagacggagatgcgaggacccctcggggtctgacccaca

SEQ ID NO: 38 - RNA, 109 nt, Homo sapiens
  source 1..109: mol_type = unassigned RNA
aagaagcttgtagatttttctcccttactatctagaattataggacttcagtccatgatttggaaaaattactggaattggagtcaaaaataatttgaaaattaggaat

SEQ ID NO: 39 - RNA, 22 nt, Homo sapiens
  source 1..22: mol_type = unassigned RNA
atgctgacatatttactagagg

SEQ ID NO: 40 - RNA, 23 nt, Homo sapiens
  source 1..23: mol_type = unassigned RNA
acttgggcactgaaacaatgtcc

SEQ ID NO: 41 - RNA, 23 nt, Homo sapiens
  source 1..23: mol_type = unassigned RNA
tgtgcttgctcgtcccgcccgca

SEQ ID NO: 42 - RNA, 22 nt, Homo sapiens
  source 1..22: mol_type = unassigned RNA
aggcggggcgccgcgggaccgc

SEQ ID NO: 43 - RNA, 22 nt, Homo sapiens
  source 1..22: mol_type = unassigned RNA
cctgacaccccatctgccctca

SEQ ID NO: 44 - RNA, 23 nt, Homo sapiens
  source 1..23: mol_type = unassigned RNA
cacacaggaaaagcggggccctg

SEQ ID NO: 45 - RNA, 21 nt, Homo sapiens
  source 1..21: mol_type = unassigned RNA
acaccctctttccctaccgcc

SEQ ID NO: 46 - RNA, 24 nt, Homo sapiens
  source 1..24: mol_type = unassigned RNA
aggaggatggagagctgggccaga

SEQ ID NO: 47 - RNA, 83 nt, Homo sapiens
  source 1..83: mol_type = unassigned RNA
ggcccggctccgggtctcggcccgtacagtccggccggccatgctggcggggctggggccggggccgagcccgcggcggggcc

SEQ ID NO: 48 - RNA, 22 nt, Homo sapiens
  source 1..22: mol_type = unassigned RNA
cgggactgtagagggcatgagc

SEQ ID NO: 49 - RNA, 80 nt, Homo sapiens
  source 1..80: mol_type = unassigned RNA
gcgtcaagatggcggcggggaggtaggcagagcaggacgccgctgctgccgccgccaccgccgcctccgctccagtcgcc

SEQ ID NO: 50 - RNA, 24 nt, Homo sapiens
  source 1..24: mol_type = unassigned RNA
tgctgatggcagatgtcgggtctg

SEQ ID NO: 51 - RNA, 22 nt, Homo sapiens
  source 1..22: mol_type = unassigned RNA
tattgcactcgtcccggcctcc

SEQ ID NO: 52 - RNA, 20 nt, Homo sapiens
  source 1..20: mol_type = unassigned RNA
gtgagtcagggtggggctgg

SEQ ID NO: 53 - RNA, 22 nt, Caenorhabditis elegans
  source 1..22: mol_type = unassigned RNA
tcaccgggtgtaaatcagcttg

SEQ ID NO: 54 - RNA, 69 nt, Homo sapiens
  source 1..69: mol_type = unassigned RNA
agaggcttgggccgccgagctggacccggaccggttttgggtactgtactgggggcagggcagagaggg

SEQ ID NO: 55 - RNA, 106 nt, Homo sapiens
  source 1..106: mol_type = unassigned RNA
gtgctcgcttcggcagcacatatactaaaattggaacgatacagagaagattagcatggcccctgcgcaaggatgacacgcaaattcgtgaagcgttccatatttt